(19) United States
(12) Patent Application Publication
(10) Pub. No.: US 2021/0185295 A1
(43) Pub. Date: Jun. 17, 2021

(54) DEVICE AND METHOD FOR ASSISTING IN 3D SCANNING A SUBJECT

(71) Applicant: KONINKLIJKE PHILIPS N.V., Eindhoven (NL)

(72) Inventors: ROBERT WILLIAM BAIKO, PITTSBURGH, PA (US); DANIEL STEED, NORTH HUNTINGDON, PA (US); RACHEL LAU, MONROEVILLE, PA (US); PRAVEEN KUMAR PANDIAN SHANMUGANATHAN, MONROEVILLE, PA (US)

(21) Appl. No.: 17/110,388

(22) Filed: Dec. 3, 2020

Related U.S. Application Data
(60) Provisional application No. 62/949,097, filed on Dec. 17, 2019.

(51) Int. Cl. H04N 13/221; G06T 7/73; H04M 1/02

(52) U.S. Cl. CPC H04N 13/221; G06T 7/73; H04M 2250/52; H04M 2250/12; H04M 1/0264

(57) ABSTRACT

A device or assisting in performing a 3D scan of a subject includes a camera structured to capture an image of the subject, an indication device structured to provide an indication, and a processing unit structured to determine a difference between a location of the camera and a desired location of the camera based on the captured image and to control the indication device to provide the indication based on the difference between the location of the camera and the desired location of the camera.

### CROSS-REFERENCE TO RELATED APPLICATIONS

This patent application claims the priority benefit under 35 U.S.C. § 119(e) of U.S. Provisional Application No. 62/949,097, filed on Dec. 17, 2019, the contents of which are herein incorporated by reference.

### BACKGROUND OF THE INVENTION

### 1. Field of the Invention

The disclosed concept relates to devices for assisting in 3D scanning of a subject. The disclosed concept also relates to methods for assisting in 3D scanning of a subject.

### 2. Description of the Related Art

Obstructive sleep apnea (OSA) is a condition that affects millions of people from around the world. OSA is characterized by disturbances or cessation in breathing during sleep. OSA episodes result from partial or complete blockage of airflow during sleep that lasts at least 10 seconds and often as long as 1 to 2 minutes. In a given night, people with moderate to severe apnea may experience complete or partial breathing disruptions as high as 200-500 per night. Because their sleep is constantly disrupted, they are deprived of the restorative sleep necessary for efficient functioning of body and mind. This sleep disorder has also been linked with hypertension, depression, stroke, cardiac arrhythmias, myocardial infarction and other cardiovascular disorders. OSA also causes excessive tiredness.

Non-invasive ventilation and pressure support therapies involve the placement of a patient interface device, which is typically a nasal or nasal/oral mask, on the face of a patient to interface the ventilator or pressure support system with the airway of the patient so that a flow of breathing gas can be delivered from the pressure/flow generating device to the airway of the patient.

Typically, patient interface devices include a mask shell or frame having a cushion attached to the shell that contacts the surface of the patient. The mask shell and cushion are held in place by a headgear that wraps around the head of the patient. The mask and headgear form the patient interface assembly. A typical headgear includes flexible, adjustable straps that extend from the mask to attach the mask to the patient.

Because patient interface devices are typically worn for an extended period of time, a variety of concerns must be taken into consideration. For example, in providing CPAP to treat OSA, the patient normally wears the patient interface device all night long while he or she sleeps. One concern in such a situation is that the patient interface device is as comfortable as possible, otherwise the patient may avoid wearing the interface device, defeating the purpose of the prescribed pressure support therapy. Additionally, an improperly fitted mask can cause red marks or pressure sores on the face of the patient. Another concern is that an improperly fitted patient interface device can include gaps between the patient interface device and the patient that cause unwanted leakage and compromise the seal between the patient interface device and the patient. A properly fitted patient interface device should form a robust seal with the patient that does not break when the patient changes positions or when the patient interface device is subjected to external forces. Thus, it is desirable to properly fit the patient interface device to the patient.

3D scanning can be employed in order to improve the fit of the patient interface device to the patient. Generally, a 3D scan can be taken of the patient's face and then the information about the patient's face can be used to select the best fitting patient interface device, to customize an existing patient interface device, or to custom make a patient interface device that fits the patient well.

Obtaining a suitable 3D scan can be difficult. Specialized 3D scanning devices are expensive and may require specialized training to operate. It is possible to generate a suitable 3D scan using a lower cost conventional 2D camera, such as those generally found on mobile phones. However, the correct techniques and positioning of the camera should be used in order to gather suitable 2D images to convert into a suitable 3D scan, which can be difficult for trained as well as untrained people.

### SUMMARY OF THE INVENTION

Accordingly, it is an object of the disclosed concept to provide a device and method that assists with capturing images for a 3D scan by providing an indication of a difference between a location of a camera and a desired location of a camera.

As one aspect of the disclosed concept, a device for performing a 3D scan of a subject comprises: a camera structured to capture an image of the subject; an indication device (104, 106, 112) structured to provide an indication; and a processing unit (102) structured to determine a difference between a location of the camera and a desired location of the camera based on the captured image and to control the indication device to provide the indication based on the difference between the location of the camera and the desired location of the camera.

As one aspect of the disclosed concept, a method for assisting with performing a 3D scan of a subject comprises: capturing an image of the subject with a camera; determining a difference between a location of the camera and a desired location of the camera based on the captured image; and providing an indication based on the difference between the location of the camera and the desired location of the camera

These and other objects, features, and characteristics of the disclosed concept, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is a view of a schematic representation of 3D scanning of a subject in accordance with an example embodiment of the disclosed concept;

FIG. 2 is a schematic diagram of an electronic device in accordance with an example embodiment of the disclosed concept;

FIG. 3 is a flowchart of a method of assisting 3D scanning of a subject in accordance with an example embodiment of the disclosed concept;

FIGS. 4A and 4B are schematic representations of providing an indication to assist with 3D scanning of a subject in accordance with an example embodiment of the disclosed concept;

FIG. 5 is a flowchart of a method of assisting with camera orientation during 3D scanning of a subject in accordance with an example embodiment of the disclosed concept;

FIGS. 6A and 6B are schematic diagrams of an electronic device including sensors to assist with camera orientation in accordance with an example embodiment of the disclosed concept;

FIG. 7 is a flowchart of a method of assisting with camera orientation during 3D scanning of a subject in accordance with an example embodiment of the disclosed concept; and

FIG. 8 is a schematic representation of aligning head and camera coordinate systems in accordance with an example embodiment of the disclosed concept.

### DETAILED DESCRIPTION OF EXEMPLARY EMBODIMENTS

As required, detailed embodiments of the disclosed concept are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the disclosed concept in virtually any appropriately detailed structure.

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

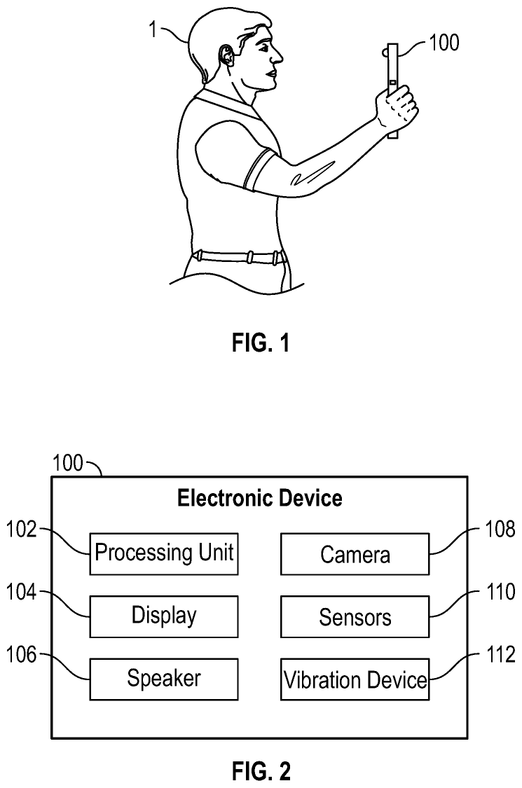

FIG. 1 is a view of a schematic representation of 3D scanning of a subject 1 in accordance with an example embodiment of the disclosed concept. 3D scanning of a subject may be accomplished with the use of an electronic device 100 having a camera. In some example embodiments, electronic device 100 may be a mobile phone with a camera. However, it will be appreciated that other types of devices capable of capturing images of subject 1 may be employed without departing from the scope of the disclosed concept.

3D scanning of subject 1 may be accomplished by capturing one or more images of subject 1 with electronic device 100. The captured images may be used to construct a 3D model of a portion of subject 1, such as subject's 1 face and/or head. Images may be captured while subject 1 holds electronic device 100 in front or to the side of him or herself. A difficulty with 3D scanning subject 1 in this manner is that the camera should be appropriately located so that subject 1 is appropriately located in the captured images. Additionally, the camera on electronic device 100 should be oriented properly with respect to subject 1 and spaced a proper distance from subject 1. In instances where the 3D scanning is performed by sweeping electronic device 100 in an arc or other pattern in front of subject 1, such sweeping should be performed at a proper speed so that images can be properly captured. Subject 1 may not be trained to position the camera of electronic device 100 properly to capture images for the 3D scan and, even if trained, it may be difficult to position the camera of electronic device 100 properly.

In accordance with an embodiment of the disclosed concept, electronic device 100 is structured to assist subject 1 in capturing images for a 3D scan by providing one or more indications to assist subject 1 with properly locating and orienting the camera of electronic device 100. Such indications may include, but are not limited to, flashing lights, colored light changes, haptic indication such as vibrations, and sounds. Such indications may change based on differences between the location and orientation of the camera of electronic device 100 and the desired location and orientation for properly capturing an image for a 3D scan of subject 1. For example, rates of sounds, vibrations, or flashing lights may increase as the desired location and/or orientation is reached. Also as an example, colored lights may change colors as the desired location and/or orientation is reached. In some examples, verbal or visual cues may be provided to direct subject to the desired location and/or orientation of electronic device 100. For example, electronic device 100 may provide a verbal cue such as “move lens up” when the camera of electronic device 100 is below the desired location for capturing an image for the 3D scan of subject 1.

In an example embodiment, different types of indications may be provided for different characteristics of the differences between the location and/or orientation of electronic device 100 and the desired location and/or orientation of the camera of electronic device 100. For example, one characteristic of the difference between the location and desired location of the camera of electronic device 100 may be a vertical difference, such as when the camera of electronic device 100 is located higher or lower than the desired location. Similarly, a horizontal difference, such as when the camera of electronic device 100 is located left or right of the desired location, may be another characteristic. Vertical orientation of the camera of electronic device 100 may be yet another characteristic. In an example embodiment, the differences between the current and desired values of these characteristics may each have their own type of indication. For example, the vertical difference may be indicated with sound, the horizontal difference may be indicated with vibration, and the vertical orientation may be indicated with flashing lights. For example, as subject 1 moves the camera of electronic device 100 vertically, reducing the vertical difference, a rate of sound (for example and without limitation, a rate of beeping) may increase, as subject 1 moves the camera of electronic device 100 horizontally, reducing the horizontal difference, a rate of vibration may increase, and as subject 1 rotates camera of electronic device 100 toward the desired vertical orientation, a rate of flashing lights may increase. Similarly, another type of indication may be used indicate a difference between the current and desired distance of the camera of electronic device 100 from subject 1. In this manner, subject 1 may be made aware of when they are approaching the desired location and/or orientation of the camera of electronic device 100 to capture an image for the 3D scan of subject 1. Subject 1 may also be made aware of which direction to move or which direction to rotate the camera of electronic device 100 to position it properly for capturing an image for the 3D scan.

FIG. 2 is a schematic diagram of electronic device 100 in accordance with an example embodiment of the disclosed concept. Electronic device 100 includes a processing unit 102, a display 104, a speaker 106, a camera 108, one or more sensors 110, and a vibration device 112. It will be appreciated by those having ordinary skill in the art that some of these components may be omitted from electronic device 100 without departing from the scope of the disclosed concept. It will also be appreciated that other components may be added to electronic device 100 without departing from the scope of the disclosed concept. In an example embodiment, electronic device 100 may be a mobile phone.

Processing unit 102 may include a processor and a memory. The processor may be, for example and without limitation, a microprocessor, a microcontroller, or some other suitable processing device or circuitry, that interfaces with the memory. The memory can be any of one or more of a variety of types of internal and/or external storage media such as, without limitation, RAM, ROM, EPROM(s), EEPROM(s), FLASH, and the like that provide a storage register, i.e., a machine readable medium, for data storage such as in the fashion of an internal storage area of a computer, and can be volatile memory or nonvolatile memory. Processing unit 102 is structured to control various functionality of electronic device 100 and may implement one or more routines stored in the memory.

Display 104 may be any suitable type of display such as, without limitation, a liquid crystal display (LCD) or a light emitting diode (LED) display. Display 104 may be structured to display various text and graphics in one or multiple colors.

Speaker 106 may be any type of device structured to emit sounds. In an example embodiment, speaker 106 is structured to selectively output sounds, such as beeps, at selected intensities and rates under control of processing unit 102.

Camera 108 is structured to capture images, such as, for example and without limitation, images of subject 1. Camera 108 may be disposed on the same side of electronic device 100 as display 104. However, it will be appreciated that camera 108 may be disposed elsewhere on electronic device 100 without departing from the scope of the disclosed concept.

Sensors 110 may include, but are not limited to, a gyrometer, an accelerometer, an angular velocity sensor, a barometer, and a pressure sensor. It will be appreciated that sensors 110 may include one or multiple of each of these types of sensors. It will also be appreciated that sensors 110 may include a limited selection of these types of sensors without departing from the scope of the disclosed concept. For example, in an embodiment, sensors 110 may include a gyrometer and an accelerometer. Similarly, in an embodiment, sensors 110 may include two pressure sensors. It will be appreciated that any number and type of sensors 110 may be employed without departing from the scope of the disclosed concept.

Vibration device 112 is structured to generate a vibration that may be used, for example, to provide haptic feedback. Vibration device 112 may be structured to selectively set and change, for example, the intensity and/or rate of vibration under control of processing unit 102.

In an embodiment, processing unit 102 is structured to receive inputs from camera 108 and/or sensors 110 and, based on said inputs, to determine a difference between the actual location and/or orientation of camera 108 and the desired location and/or orientation of camera 108 for capturing images for a 3D scan of subject 1. For example, processing unit 102 may receive images captured with camera 108 as an input, and, based on said images, determine the difference between the location of camera 108 and the desired location of camera 108. Processing unit 102 may, for example, identify subject 1 in a captured image and determine a difference between where subject 1 is located in the captured image and the desired location of subject in the captured image. As part of the process, processing unit 102 may, for example, identify landmarks in the captured image of subject 1, such as the tip of the nose.

Similarly, processing unit 102 may receive inputs from sensors 110, and, based on said inputs, determine the difference between the orientation of camera 108 and the desired orientation of camera 108. For example, in an embodiment, a vertically oriented camera 108 may be desired, and, based on inputs from sensors 110, processing unit 102 may determine the difference between the orientation of camera 108 and the desired orientation.

Processing unit 102 is further structured to control indication devices such as display 104, speaker 106, and/or vibration device 112 to provide indications based on the difference between the actual location and/or orientation of camera 108 and the desired location and/or orientation of camera 108. Such indications may include, but are not limited to, flashing lights, colored light changes, vibrations, and sounds. Such indications may change based on differences between the location and orientation of camera 108 and the desired location and orientation for properly capturing an image for a 3D scan of subject 1. For example, rates of sounds, vibrations, or flashing lights may increase as the desired location and/or orientation is reached. Also as an example, colored lights may change colors as the desired location and/or orientation is reached. In some examples, verbal or visual cues may be provided to direct subject to the desired location and/or orientation of camera 108. For example, electronic device 100 may provide a verbal cue such as “move lens up” when camera 108 is below the desired location for capturing an image for the 3D scan of subject 1.

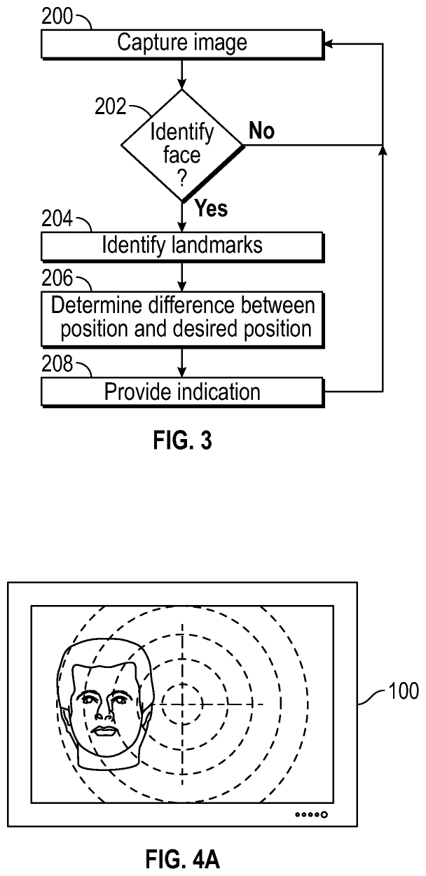

FIG. 3 is a flowchart of a method of assisting 3D scanning of subject 1 in accordance with an example embodiment of the disclosed concept. The method of FIG. 3 may be implemented, for example, with electronic device 100. The method begins at 200 with capturing an image of subject 1. The image may be captured, for example, using camera 108. At 202, it is determined whether a face is identified in the captured image. For example, processing unit 102 may use any suitable facial recognition technique to determine whether a face is present in the captured image. If no face is present, the method returns to 200 where another image is captured. Images may continue to be captured until a face is recognized in a captured image. Once a face is recognized, the method proceeds to 204.

At 204, one or more landmarks are identified in the image of the captured face. The one or more landmarks may, for example, be easily identifiable features of the face. For example, the tip of the nose may be a landmark that is identified. However, it will be appreciated that other landmarks may be used without departing from the scope of the disclosed concept. At 206, the difference between the position of subject 1 in the captured image and the desired position of subject 1 in the captured image is determined. For example, the location of the landmark (e.g. the tip of the nose) may be compared to a desired location of the landmark in the captured image. In an example, the desired location of the tip of the nose is the center of the captured image. However, it will be appreciated that the landmark and the desired location of the landmark may be different without departing from the scope of the disclosed concept.

Once the difference between the actual position and desired position has been determined, the method proceeds to 208 where an indication is provided. The indication may be any of the previous indications described herein. As described herein, the indication may change based on the magnitude of the difference between the actual location and desired location. The method then returns to 200. The method may continuously run as the subject 1 locates and orients electronic device 1 while images are captured for a 3D scan of subject 1. The continuously updated indications assist subject 1 in properly locating electronic device 100 for capturing images during the 3D scan.

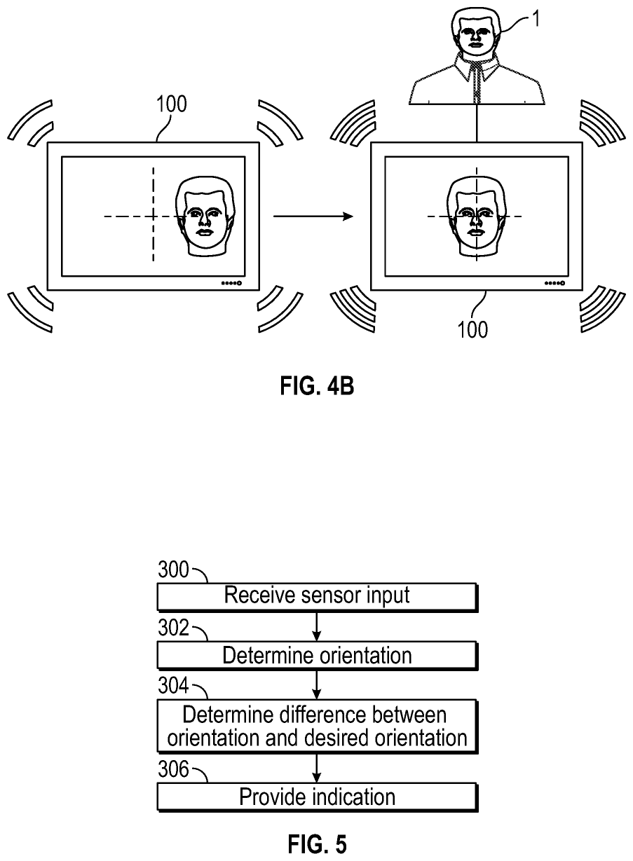

FIGS. 4A and 4B are schematic representations of providing an indication to assist with 3D scanning of subject 1 in accordance with an example embodiment of the disclosed concept. FIGS. 4A and 4B are illustrative example of the method described with respect to FIG. 3. FIG. 4A illustrates an example of an image of subject 1 captured by electronic device 100. As shown in FIG. 4A, the captured image of subject 1 is off-center.

In FIG. 4B, in the left image, the captured image of subject 1 is off-center and electronic device 100 provides an audible indication at a first rate. In the right image, the location of electronic device 100 has been adjusted so that subject 1 is centered in the captured image. Based on electronic device 100 and camera 108 being moved to the desired location, the rate of the audible indication has been increased in order to indicate to subject 1 that the desired location has been reached. In this manner, subject 1 can properly locate electronic device 100 and camera 108 to capture images for the 3D scan.

FIG. 5 is a flowchart of a method of assisting with orientation during 3D scanning of subject 1 in accordance with an example embodiment of the disclosed concept. The method FIG. 5 may be implemented, for example, with electronic device 100. The method begins at 300, where sensor input is received. The sensor input may be received, for example, from one or more of sensors 110. The sensor input may be indicative of the orientation of electronic device 100. At 302, the orientation of electronic device 100 is determined based on the sensor input. The method then proceeds to 304 where a difference between the orientation and the desired orientation is determined. In some examples, a vertical orientation is the desired orientation. However, it will be appreciated that other orientations may be desired based on the application. Once the difference between the orientation and the desired orientation is determined, an indication is provided at 306 based on the difference. As described herein, the indication may change based on the magnitude of the difference. Furthermore, one type of indication may be provided based on the difference between the actual and desired orientation and another type of indication may be provided based on the difference between the actual and desired location.

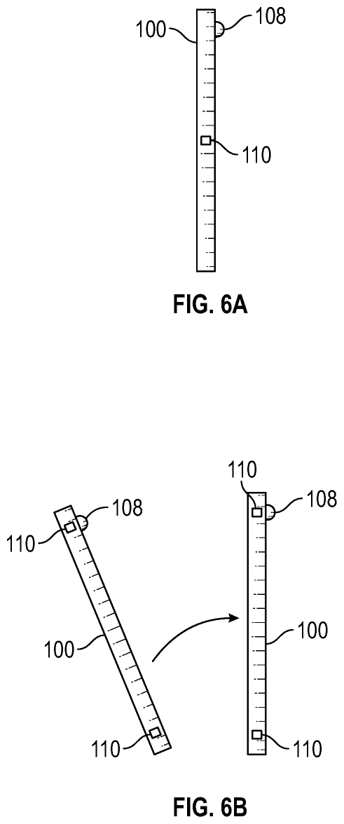

FIGS. 6A and 6B are schematic diagrams of electronic device 100 including sensors 110 to assist with camera orientation in accordance with an example embodiment of the disclosed concept. In the example shown in FIG. 6A, electronic device 100 includes sensor 110 that senses the vertical orientation of electronic device 100 and camera 108. For example, sensor 110 may be a gyrometer, accelerometer, or angular velocity sensor.

In the example shown in FIG. 6B, electronic device 100 incudes sensors 110 that are pressure sensors disposed at opposite ends of electronic device 100. Processing unit 102 may determine the vertical orientation of electronic device 100 and camera 108 based on the difference between pressures detected by sensors 110. When electronic device 100 and camera 108 are moved from a non-vertical orientation, shown on the left side of FIG. 6B, to a vertical orientation, shown in the right side of FIG. 6B, the difference between the pressures sensed by the sensors 110 will increase. When electronic device 100 and camera 108 are vertically oriented, the difference in pressures sensed by sensors 110 will be at a maximum value.

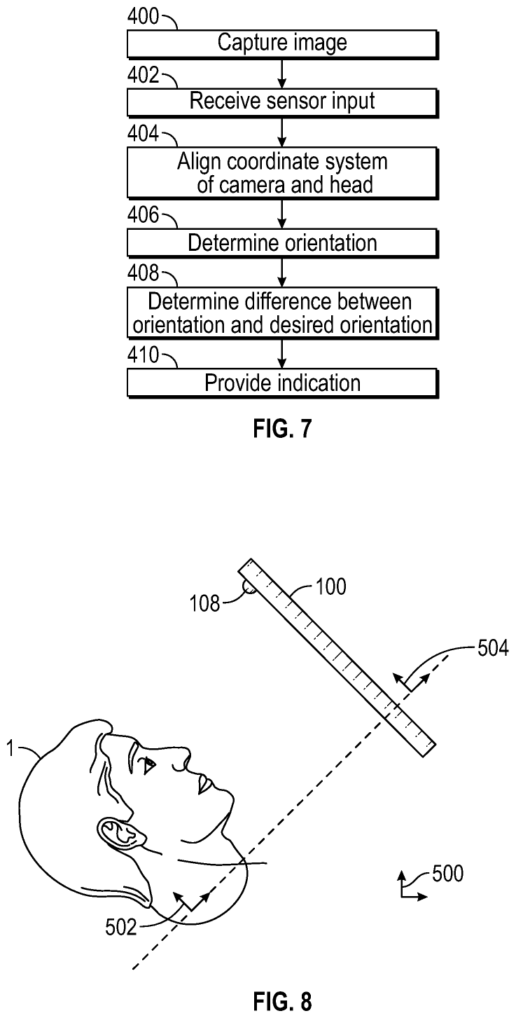

FIG. 7 is a flowchart of a method of assisting with orientation during 3D scanning of subject 1 in accordance with an example embodiment of the disclosed concept. The method of FIG. 7 may be implemented, for example, with electronic device 100. When subject's 1 face is vertically oriented, a vertical orientation of electronic device 100 is desirable for capturing images for a 3D scan. However, subject 1 often will not be vertically oriented themselves. For example, subject 1 may be looking upward or downward. In this case, electronic device 100 and camera 108 should be oriented based on the orientation of subject's 1 face. The method described with respect to FIG. 7 assists subject 1 with orienting electronic device 100 and camera 108 in this situation.

The method begins at 400 where an image of subject 1 is captured. At 402, sensor input is received. The sensor input is indicative of the orientation of electronic device 100 and camera 108 and may be received, for example, by processing unit 102 from sensors 110. At 404, the coordinate system of electronic device 100 and camera 108 is aligned with the coordinate system of subject's 1 head. For example, as shown in FIG. 8, subject's 1 head is tilted backward. Coordinate system 502 system of subject's 1 head is thus different than a local coordinate system 500. That is, the vertical axis in coordinate system 502 of subject's 1 head is based on the orientation of subject's 1 head rather than the vertical axis in local coordinate system 500. Coordinate system 504 of electronic device 100 and camera 108 is aligned with coordinate system 502 of subject's 1 head. When coordinate systems 502,504 are aligned, the vertical orientation of electronic device 100 and camera 108 in the aligned coordinate system 504 is desired, even though it is different than vertical orientation in local coordinate system 500. This is because the vertical orientation of electronic device 100 and camera 108 in the aligned coordinate system 504 is aligned with subject's 1 head so that camera 108 will be facing straight toward subject's 1 head rather than being at a skewed angle with respect to subject. Alignment of the coordinate systems may be performed, for example, by analyzing the captured image of subject 1.

Once the coordinate system of electronic device 100 and camera 108 is aligned with the coordinate system of subject's 1 head, the method proceeds to 406, where the orientation of electronic device 100 and camera 108 is determined. The orientation may be determined based on inputs from sensors 110, as has been described herein. At 408, a difference between the orientation of electronic device 100 and camera 108 and the desired orientation of electronic device 100 and camera 108 is determined. The desired orientation may be, for example, a vertical orientation in the aligned coordinate system 504. At 410, an indication is provided at based on the difference between the orientation of electronic device 100 and camera 108 and the desired orientation. As described herein, the indication may change based on the magnitude of the difference. Furthermore, one type of indication may be provided based on the difference between the actual and desired orientation and another type of indication may be provided based on the difference between the actual and desired location. In this manner, subject 1 may be assisted in properly aligning electronic device 100 to capture images for a 3D scan even when subject's 1 head is tilted.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

What is claimed is:

1. A device for performing a 3D scan of a subject, the device comprising:
a camera structured to capture an image of the subject;
an indication device structured to provide an indication; and
a processing unit structured to determine a difference between a location of the camera and a desired location of the camera based on the captured image and to control the indication device to provide the indication based on the difference between the location of the camera and the desired location of the camera.

2. The device of claim 1, wherein the processing unit is structured to determine the difference between the camera and a desired location of the camera by determining a difference between a location of subject in the captured image with a desired location of subject in the captured image.

3. The device of claim 2, wherein the processing unit is structured to determine one or more landmarks of the subject in the captured image.

4. The device of claim 1, wherein the indication is at least one of a haptic and an audible indication.

5. The device of claim 1, wherein the indication is a visual indication.

6. The device of claim 1, wherein the processing unit is structured to determine a rate of the indication based on a magnitude of the difference between the location of the camera and the desired location of the camera and to control the indication device to provide the indication at the determined rate.

7. The device of claim 1, further comprising one or more sensors structured to sense an orientation of the camera, wherein the processing unit is structured to determine a difference between the orientation of the camera and a desired orientation of the camera based on an output of the one or more sensors and to control the indication device to provide the indication based on the difference between the orientation of the camera and the desired orientation of the camera.

8. The device of claim 7, wherein the indication includes a first indication type and a second indication type, wherein the processing unit is structured to control the indication device to provide the first indication type based on the difference between the location of the camera and the desired location of the camera and to provide the second indication type based on the difference between the orientation of the camera and the desired orientation of the camera.

9. The device of claim 7, wherein the one or more sensors include a first pressure sensor and a second pressure sensor, wherein the processing unit is structured to determine the orientation of the camera based on a difference between outputs of the first pressure sensor and the second pressure sensor.

10. The device of claim 7, wherein the one or more sensors include at least one of a gyrometer, an accelerometer, and an angular velocity sensor.

11. A method for assisting with performing a 3D scan of a subject, the method comprising:
capturing an image of the subject with a camera;
determining a difference between a location of the camera and a desired location of the camera based on the captured image; and
providing an indication based on the difference between the location of the camera and the desired location of the camera.

12. The method of claim 12, wherein device of claim 1, wherein determining the difference between a location of the camera and the desired location of the camera based on the captured image includes determining a difference between a location of subject in the captured image with a desired location of subject in the captured image.

13. The method of claim 12, further comprising determining a rate of the indication based on a magnitude of the difference between the location of the camera and the desired location of the camera, wherein providing the indication includes providing the indication at the determined rate.

14. The method of claim 12, further comprising:
receiving outputs of one or more sensors structured to sense an orientation of the camera; and
determining a difference between the orientation of the camera and a desired orientation of the camera based the outputs of the one or more sensors, wherein providing the indication includes providing the indication based on the difference between the orientation of the camera and the desired orientation of the camera.

15. The method of claim 14, wherein the indication includes a first indication type and a second indication type, wherein providing the indication includes providing the first indication type based on the difference between the location of the camera and the desired location of the camera and providing the second indication type based on the difference between the orientation of the camera and the desired orientation of the camera.
